(19) United States
(12) Patent Application Publication
(10) Pub. No.: US 2025/0346846 A1
(43) Pub. Date: Nov. 13, 2025

(54) SINGLE-USE BIOREACTOR INTERFACES FOR SINGLE-USE PROBE

(71) Applicant: Merck Patent GmbH, Darmstadt (DE)

(72) Inventors: Celia Sanchez, Grenoble (FR); Celine Duchemin, Montbonnot Saint Martin (FR); Renaud Puget, Bernin (FR); Fabrice Thomas, Saint Martin D'Heres (FR); Laurent Jourdainne, Stutzheim-Offenheim (FR)

(21) Appl. No.: 18/867,553

(22) PCT Filed: May 25, 2023
(86) PCT No.: PCT/US2023/023541

§ 371 Date: Nov. 20, 2024

Related U.S. Application Data
(60) Provisional application No. 63/347,683, filed on Jun. 1, 2022.

(51) Int. Cl. C12M 1/00; C12M 3/00

(52) U.S. Cl. CPC C12M 23/28; C12M 23/14; C12M 23/22; C12M 23/38; C12M 23/48

(57) ABSTRACT

An optical interface with a housing such as a bioreactor bag, and a bioreactor bag having an optical interface. In some embodiments, the optical principle of Raman technology is used to provide several embedded single-use optical interfaces in a housing, with associated Raman probe modifications if necessary. In some embodiments, the optical interface includes a single-use cap to reduce the ambient light intensity which can be detrimental to a Raman signal. The device in accordance with embodiments disclosed herein allows for the avoidance of sterility breaks that typically may occur due to the insertion into the housing of an intrusive reusable sensor through an aseptic connection.

### RELATED APPLICATIONS

The present application claims the benefit of U.S. Provisional Patent Application No. 63/347,683, filed on Jun. 1, 2022, the entire contents of which is incorporated by reference herein in its entirety.

### BACKGROUND

Single-use disposable bioreactors are often the vessel of choice for biomolecule processing. Bags are often used as such single-use containers to receive and maintain a fluid, such as a biological fluid. The bag may comprise monolayer walls or multilayer flexible walls formed of a polymeric composition such as polyethylene, including ultrahigh molecular weight polyethylene (UHMWPE), ultralow density polyethylene (ULDPE), linear low density polyethylene (LLDPE), low density polyethylene (LDPE), medium density polyethylene (MDPE): polypropylene (PP): ethylene vinyl alcohol (EVOH): polyvinyl chloride (PVC): polyvinyl acetate (PVA): ethylene vinyl acetate copolymers (EVA copolymers); thermoplastic elastomers (TPE), and/or blends or alloys of any of the foregoing materials as well as other various thermoplastics materials and additives as are known to those in the art. The bag should be capable of supporting a biologically active environment, such as one capable of growing cells in the context of cell cultures. The bag may be a two-dimensional (2D) or “pillow” bag or may be a three-dimensional (3D) bag. The bag may include one or more inlets, one or more outlets and, optionally, other features such as sterile gas vents, spargers, and ports for the sensing of the liquid within the container for parameters such as conductivity, pH, temperature, dissolved gases, e.g., oxygen and carbon dioxide, and the like as known to those in the art.

Conventional monitoring strategies of mammalian cell culture lack real-time measurement and rely on daily or other periodic manual sampling and feeding, which increases the risk of contamination and batch failures. Raman analyzers, such as the ProCellics™ Raman Analyzer with Bio4C™ PAT Raman software, available from Merck KGaA, enables the performance of in-line and real-time monitoring, assessing and/or measurement of various parameters of bioreactor cultures. These parameters may include one or more critical process parameters such as concentrations of glucose, lactate and ammonium, as well as key performance indicators such as total cell density and viable cell density. However, the sensing apparatus, which may or may not be single-use or disposable, must not deleteriously interfere with or compromise the integrity of the single-use device. In addition, the manner in which the sensing apparatus communicates with the contents of the bioreactor may be problematic on multiple levels, including sterility, compatibility of components, manner of attachment and detachment, etc.

Raman analyzers and instruments are mainly multi-use and require an autoclave process or the like for sterilization before being integrated into a bioreactor bag. That also requires a specific aseptic interface for the probe insertion in the bag.

It would be desirable to provide a single-use analyzer optical interface with a bioreactor bag in order to improve current systems.

For further understanding of the nature of these and other objects of the present disclosure, reference should be had to the following description considered in conjunction with the accompanying drawings.

### SUMMARY

Problems of the prior art have been addressed by embodiments disclosed herein, which relate to an optical interface with a container or housing, e.g., a single-use bioreactor and/or a mixer single-use bag. In some embodiments, the optical principle of Raman technology is used to provide several embedded single-use optical interfaces in a bioreactor bag, with associated Raman probe modifications if necessary. Preferably the interface is gamma sterilization compatible. In some embodiments, the optical interface includes a single-use cap to reduce the ambient light intensity which can be detrimental to a Raman signal. The device in accordance with embodiments disclosed herein allows for the avoidance of sterility breaks that typically may occur due to the insertion into the bioreactor of an intrusive reusable sensor through an aseptic connection.

In some embodiments, the optical interface may include a single-use cap to reduce the ambient light intensity which can be detrimental to the Raman signal.

In certain embodiments, the optical interface includes a sleeve configured to receive a probe, such as the sampling immersion tube of a Raman probe, the sleeve having a light-reducer such that external light that can interfere with Raman signal generation is decreased or eliminated. In some embodiments, the sleeve cooperates with a barb that interfaces with the bioreactor, and a clamp or the like that secures the sleeve to the barb. In certain embodiments the interface is leakproof even if there is no probe present, as the interface may be sterilized and keeps the container or housing (e.g., the bag) closed even in the absence of a probe.

In some embodiments, the sleeve includes an optical window, such as a sapphire or quartz window. Consequently, the probe itself can be devoid of such a window.

Accordingly, disclosed is an interface assembly for insertion of a probe into a bioreactor, the interface assembly comprising an elongated sleeve having a passageway configured to receive the probe, the sleeve containing a window; and a clamp for fastening the sleeve to a fitting positionable in a port of the bioreactor. In some embodiments, the passageway has a generally cylindrical cross-section. In some embodiments, the sleeve has a proximal end where the probe is inserted, and a distal end spaced from the proximal end, and wherein a cap is positioned at the distal end. The cap may extend longitudinally from the window, and may include one or more radial openings. In some embodiments, the interface further comprises a barb fitting having a barbed stem that secures to the clamp, and a flange configured to be positioned in an interior volume of the bioreactor. In some embodiments, the window is a sapphire or quartz window.

In some embodiments, disclosed is a bioreactor having an internal volume, the bioreactor comprising a port providing access to the internal volume from a region external to the bioreactor, and a fitting attached to the port, the fitting being configured to receive an interface assembly configured to receive a probe into the bioreactor volume through the port, the interface assembly comprising an elongated sleeve having a passageway configured to receive the probe, the sleeve containing a window such as a sapphire or quartz window. The interface assembly may further comprise a clamp for fastening the sleeve to the fitting, and the fitting may be a barbed fitting or may be a fitting threaded to the port.

Accordingly, disclosed is an interface assembly configured for insertion of a probe into a housing such as a bioreactor or bioreactor bag, the interface assembly comprising an elongated sleeve having a passageway configured to receive the probe, the sleeve containing an optical window; and a clamp for fastening the sleeve to a fitting positionable in a port of the housing. The sleeve may have a proximal end where the probe is inserted, and a distal end spaced from the proximal end, wherein a cap is positioned at the distal end. The cap may extend longitudinally from the window, and may have one or more radial openings. The probe may include an optical lens positioned next to the window of the sleeve when in the assembled condition. The interface assembly may further comprise a barb fitting having a barbed stem that secures to the clamp, and a flange configured to be positioned in an interior volume of the housing. The housing may have a threaded port such that the interface assembly threads to the threaded port. The elongated sleeve may be a single-use sleeve. The optical window may comprise sapphire.

Also disclosed is a housing having an internal volume, the housing comprising a port providing access to the internal volume from a region external to the housing, and a fitting attached to the port, the fitting being configured to receive an interface assembly configured for insertion of a probe into the volume through the port, the interface assembly comprising an elongated sleeve having a passageway configured to receive the probe, the sleeve containing an optical window. The interface assembly may further comprise a clamp for fastening the sleeve to the fitting, which may be a barbed fitting and may be threaded to the port. The optical window may comprise sapphire. The housing may be a bioreactor or bioreactor bag.

### BRIEF DESCRIPTION OF THE DRAWINGS

FIG. 1 is a perspective view of an optical interface according to some embodiments of the disclosure:

FIG. 2A is a perspective view of an optical interface inserted into a barb fitting according to some embodiments of the disclosure;

FIG. 2B is a cross-sectional view of an optical interface inserted into a barb fitting according to some embodiments of the disclosure:

FIG. 3 is a cross-sectional view of an optical interface with a probe tube inserted therein according to some embodiments of the disclosure:

FIG. 4 is a perspective view of an optical interface with a probe tube inserted therein according to some embodiments of the disclosure:

FIG. 5A is a perspective view of a probe and an optical interface prior to insertion of the probe into the interface according to some embodiments of the disclosure:

FIG. 5B is a perspective view of a probe and an optical interface after insertion of the probe into the interface according to some embodiments of the disclosure;

FIG. 6A is a perspective view of a probe and an alternative embodiment of an optical interface prior to insertion of the probe into the interface according to some embodiments of the disclosure:

FIG. 6B is a perspective view of a probe and an alternative embodiment of an optical interface after insertion of the probe into the interface according to some embodiments of the disclosure:

FIG. 7A is a cross-sectional view of a probe and the alternative embodiment of an optical interface of FIG. 6A according to some embodiments of the disclosure:

FIG. 7B is a perspective view of the probe and alternative embodiment of an optical interface of FIG. 7A according to some embodiments of the disclosure:

FIG. 8 is a perspective view of a still further embodiment of an optical interface according to some embodiments of the disclosure:

FIG. 9A is a perspective view of yet a further embodiment of an optical interface according to some embodiments of the disclosure;

FIG. 9B is a perspective view of the embodiment of FIG. 9A including a probe just prior to insertion into an optical interface according to some embodiments of the disclosure;

FIG. 9C is a perspective view of the embodiment of FIG. 9A including a probe shown inserted into an optical interface according to some embodiments of the disclosure;

FIG. 9D is a cross-sectional view of the optical interface of FIG. 9A according to some embodiments of the disclosure;

FIG. 10 is a perspective view of an additional embodiment of an optical interface shown engaged with a probe according to some embodiments of the disclosure;

FIG. 11 is a perspective view of a bioreactor, including an exploded view of an optical interface prior to insertion into a barb fitting on a port of the bioreactor, according to some embodiments of the disclosure;

FIG. 12 is a perspective view of the bioreactor of FIG. 11 shown with the optical interface inserted into a barb fitting on a port of the bioreactor, according to some embodiments of the disclosure:

FIG. 13 is a perspective view of a bioreactor, including an exploded view of an optical interface prior to insertion into a threaded port of the bioreactor, according to some embodiments of the disclosure; and

FIG. 14 is a perspective view of the bioreactor of FIG. 13 shown with the optical interface inserted into the threaded fitting on a port of the bioreactor, according to some embodiments of the disclosure.

### DETAILED DESCRIPTION

A more complete understanding of the components, processes and apparatuses disclosed herein can be obtained by reference to the accompanying drawing. The figures are merely schematic representations based on convenience and the ease of demonstrating the present disclosure, and is, therefore, not intended to indicate relative size and dimensions of the devices or components thereof and/or to define or limit the scope of the exemplary embodiments.

Although specific terms are used in the following description for the sake of clarity, these terms are intended to refer only to the particular structure of the embodiments selected for illustration in the drawing, and are not intended to define or limit the scope of the disclosure. In the drawing and the following description below, it is to be understood that like numeric designations refer to components of like function.

The singular forms “a,” “an,” and “the” include plural referents unless the context clearly dictates otherwise.

As used in the specification, various devices and parts may be described as “comprising” other components. The terms “comprise(s),” “include(s),” “having,” “has,” “can,” “contain(s),” and variants thereof, as used herein, are intended to be open-ended transitional phrases, terms, or words that do not preclude the possibility of additional components.

All ranges disclosed herein are inclusive of the recited endpoint and independently combinable (for example, the range of “from 2 inches to 10 inches” is inclusive of the endpoints, 2 inches and 10 inches, and all the intermediate values).

As used herein, approximating language may be applied to modify any quantitative representation that may vary without resulting in a change in the basic function to which it is related. Accordingly, a value modified by a term or terms, such as “about” and “substantially,” may not be limited to the precise value specified, in some cases. The modifier “about” should also be considered as disclosing the range defined by the absolute values of the two endpoints. For example, the expression “from about 2 to about 4” also discloses the range “from 2 to 4.”

It should be noted that many of the terms used herein are relative terms. For example, the terms “upper” and “lower” are relative to each other in location, i.e. an upper component is located at a higher elevation than a lower component, and should not be construed as requiring a particular orientation or location of the structure. As a further example, the terms “interior”, “exterior”, “inward”, and “outward” are relative to a center, and should not be construed as requiring a particular orientation or location of the structure.

The terms “top” and “bottom” are relative to an absolute reference, i.e. the surface of the earth. Put another way, a top location is always located at a higher elevation than a bottom location, toward the surface of the earth.

The terms “horizontal” and “vertical” are used to indicate direction relative to an absolute reference, i.e. ground level. However, these terms should not be construed to require structures to be absolutely parallel or absolutely perpendicular to each other.

The terms “bioreactor,” “bag,” and “container” are generally used interchangeably within this disclosure. The terms bioreactor, bag and container as used herein refers to any manufactured or engineered device or system that supports a biologically active environment, including mixer bags such as bags for cell culture media preparation, antibody drug conjugate (ADC) reaction, or other downstream processing steps that are or may be carried out in bulk. In some instances, a bioreactor is a vessel having an inner volume in which a cell culture process is carried out which involves organisms or biochemically active substances derived from such organisms. A flexible bioreactor, bag, or container connotes a flexible vessel that can be folded, collapsed, and expanded and/or the like, capable of containing, for example, a biological fluid. A single use bioreactor, bag, or container, typically also flexible, is a vessel that is used once and discarded. In some embodiments, the bag, bioreactor or container may be a two-dimensional (2D) or “pillow” bag or, alternatively, may be a three-dimensional (3D) bag. The particular geometry of the container or bioreactor is not limited in any embodiment disclosed herein. In some embodiments, the container may include a rigid base, which provides access points such as ports or vents. Any container described herein may comprise one or more inlets, one or more outlets and, optionally, other features such as sterile gas vents, spargers, and ports for the sensing of the liquid within the container for parameters such as conductivity, pH, temperature, dissolved gases, e.g., oxygen and carbon dioxide, and the like as known to those in the art. The container is of a sufficient size to contain fluid. such as cells and a culture medium, to be mixed from pilot scale, e.g., 50 L to small or to large production volume containers, e.g., 500 L to 3000 L or larger bioreactors.

The bioreactor may comprise a flexible and collapsible bag having an interior surface and an exterior surface. Interior surface bounds a compartment or space into which liquid can be added. The bag may comprise a side wall that, when the bag is unfolded, has a substantially circular or polygonal transverse cross section that extends between a first end and an opposing second end. First end terminates at a top end wall while the second end terminates at a bottom end wall. The bag may be comprised of a flexible, water impermeable material such as polyethylene or other polymeric sheets having a thickness in a range between about 0.1 mm to about 5 mm with about 0.2 mm to about 2 mm being more common. Other thicknesses also may be used. The material can be comprised of a single ply material or can comprise two or more layers which are either sealed together or separated to form a double wall container. Where the layers are sealed together, the material can comprise a laminated or extruded material. The laminated material comprises two or more separately formed layers that are subsequently secured together by an adhesive. The bag material comprises a single integral sheet which comprises two or more layer of different material separated by a contact layer that are either laminated together or all simultaneously co-extruded. In one embodiment, the material is approved for direct contact with living cells and is capable of maintaining a solution sterile. In such an embodiment, the material can also be sterilizable such as by ionizing radiation. Other examples of materials that can be used are disclosed in U.S. Pat. No. 6,083,587 which issued on Jul. 4, 2000 and U.S. patent application Ser. No. 10/044,636, filed Oct. 19, 2001 which are hereby incorporated by specific reference.

The terms “sterile” and “sterilized” are defined as a condition of being free from contaminants and, particularly within the bioprocessing industry, free from pathogens, such as undesirable viruses, bacteria, germs, and other microorganisms. Relatedly, the terms “bioburden-reduced” and “bioburden reduction” (e.g., by a non-sterilizing dose of gamma or X-ray radiation<25 kGy) may be substituted for certain embodiments that do not necessitate a sterile claim.

In certain embodiments, disclosed is a bioreactor system that comprises a bioreactor volume or chamber for containing a bioreactor culture and a probe capable of providing a Raman spectrum of the bioreactor culture in the bioreactor volume or chamber. In some embodiments, the probe interfaces with the bioreactor chamber with the optical interface described herein.

In certain embodiments, a computer comprising a processor or processing unit which executes computer readable instructions, may be provided in the bioreactor system. The processor or processing unit may be a general purpose computing device, such as a microprocessor. Alternatively, it may be a specialized processing device, such as a programmable logic controller (PLC). A storage element also may be provided, which is used to store instructions, as well as provide temporary storage for the processor's use. The storage element may utilize any memory technology, such as RAM, ROM, EEPROM, Flash ROM, NVRAM, or any other suitable technology. An input device may be provided, such as a touchscreen, keyboard, or other suitable device.

In some embodiments, Raman spectroscopy may be performed in the visible, near infrared, infrared, near ultraviolet, or ultraviolet (UV) range. In some embodiments, Surface Enhanced Raman Spectroscopy (SERS) may be used. In some embodiments, resonance Raman spectroscopy, tip-enhanced Raman spectroscopy, polarized Raman spectroscopy, stimulated Raman spectroscopy, transmission Raman spectroscopy, spatially offset Raman spectroscopy, difference Raman spectroscopy, Fourier Transform (FT) Raman, or hyper Raman spectroscopy may be used.

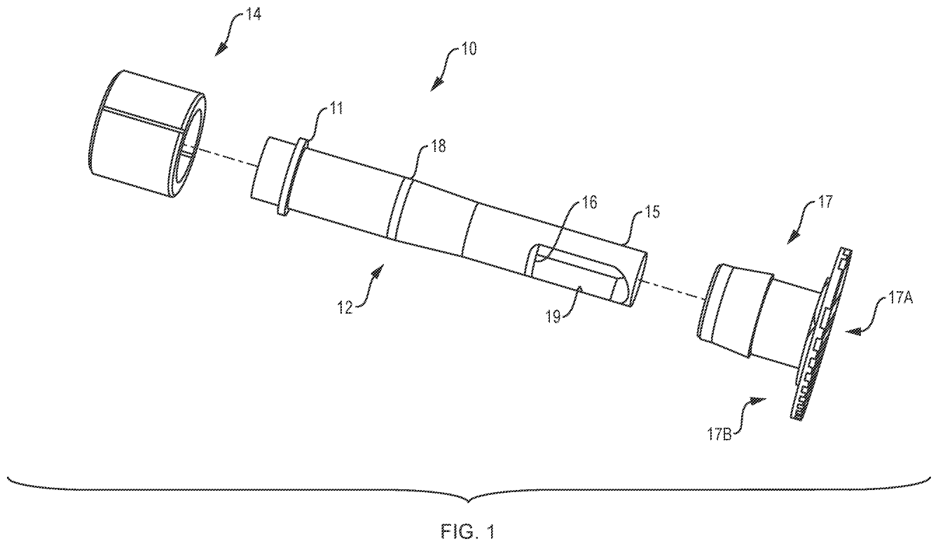

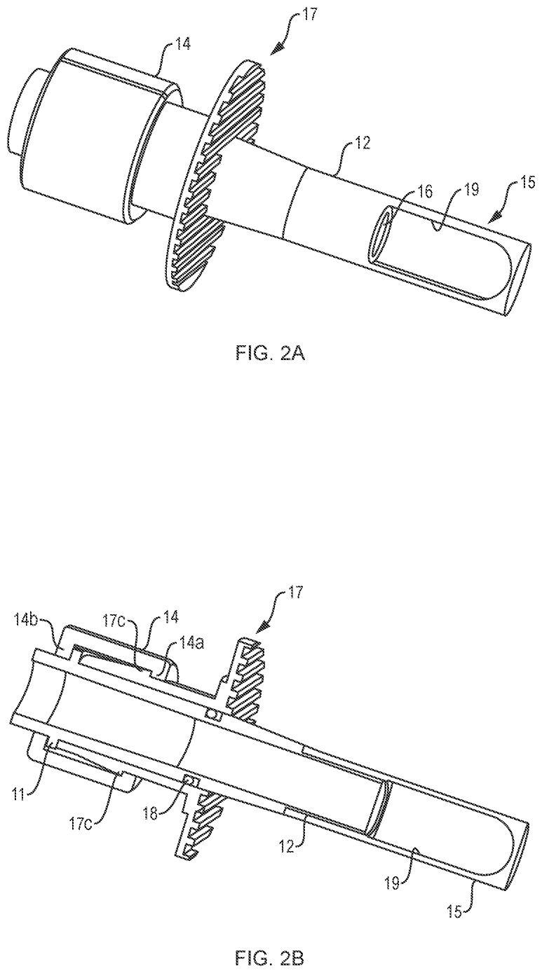

Turning now to FIGS. 1, 2A and 2B, there is shown an optical interface assembly 10 in accordance with certain embodiments. In some embodiments, the optical interface assembly 10 includes a sleeve 12 and a clamp 14. The sleeve 12 may have a generally cylindrical configuration and an internal bore or passageway configured (e.g., shaped and dimensioned) to receive a probe, such as a Raman probe. In the embodiment shown, the sleeve 12 includes an optional cap 15 positioned at the distal end of the sleeve 12 which may be integral to the sleeve 12. The cap 15 has one or more radial openings or through-holes 19 that terminate prior to the distal-most end of the cap 15. The one or more radial openings may provide access to or expose the optical window to the contents of the bioreactor when in the assembled condition. The cap 15 helps to manage stray light (e.g., reduce ambient light intensity that may interfere with the Raman signal) and helps to protect the user from laser light that may otherwise escape from the sleeve 12. An optical window 16 may be positioned at or near the end of the passageway as shown, such as an overmolded sapphire window (as is known by those skilled in the art, overmolding is a process of adding an additional layer of material over an already existing piece or part. Typically the first material, sometimes referred to as the substrate, is partially or fully covered by subsequent materials (overmold materials) during the manufacturing process). Those skilled in the art will appreciate that any material suitable for the application may be used for the optical window, such as sapphire or quartz.

Preferably the sleeve 12 is single-use, i.e., disposable; designed to be discarded after one use. Preferably it is made of a gamma compatible material for ease of sterilization, such as a plastic or stainless steel, including plastic materials with a Raman signature with low intensities such as for example plastic with CxHy composition, e.g. polyethylene (PE), polypropylene (PP), polystyrene (PS), polystyrene sulfonate (PSS), polycarbonate (PC), polyamide PA6/PA12, etc.

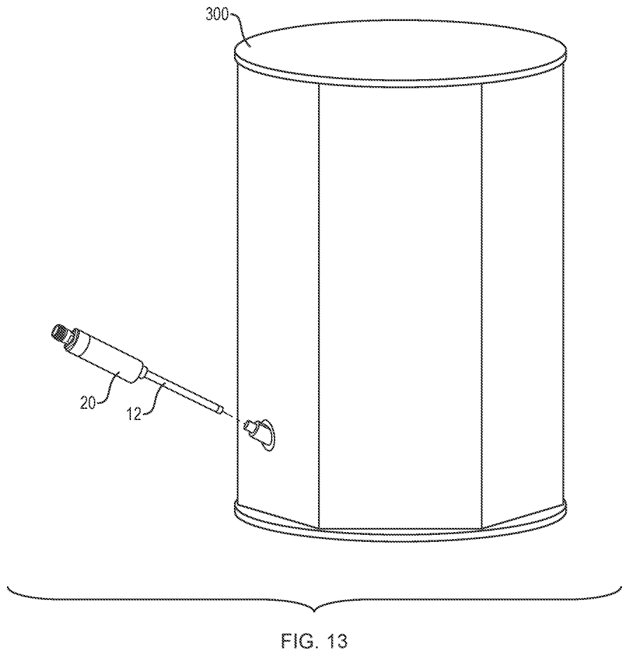

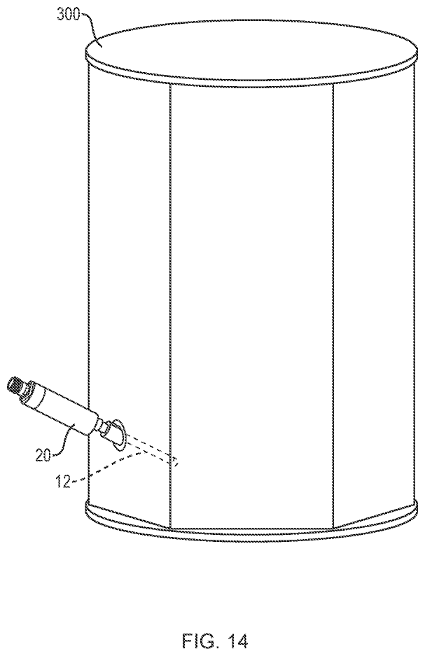

The outside diameter of the sleeve 12 is configured to receive a standard hose barb 17, such as a 1″ barb. The barb 17 is configured to mate, dock, couple or otherwise integrate, typically mechanically, with the bioreactor bag 300 such as shown in FIGS. 11 and 12, in a conventional manner, such as with a port of the bag, and includes a barbed stem that secures to clamp 14. Thus, the barb 17 has a first region 17A such as a flange that in use is positioned in the bioreactor chamber or volume; i.e., is internal thereto, and a second region 17B such as a stem with one or more radial ridges or serrations and is external to the bag: e.g., a probe insertion side. The flange encircles and is radially outwardly projecting from the stem. The barb 17 may include threads, such as internal threads, to secure or maintain the probe in the sleeve 12. Another embodiment is shown in FIGS. 13 and 14, where a PG13.5 or other thread on the bag 300 is used, and the single-use sleeve 12 is threaded thereto. FIG. 14 shows the insertion of the tube and connection of the probe to the bag 300 in this embodiment. The threaded member coupled to the bioreactor port may include a sapphire window and also may include an optical lens.

In certain embodiments, the sleeve 12 is oriented for insertion in the barb 17, as shown in FIG. 2B, and secures to the barb 17 with a fastener such as clamp 14 (FIGS. 2A, 2B). In some embodiments, the clamp 14 includes a distal annular radially inwardly projecting ridge 14A which abuts against the barb radial ridge 17C on the stem and locks the barb in place, and a proximal annular radially inwardly projecting ridge 14B which abuts against an annular ring 11 on the sleeve 12. The annular ring 11 acts as a stop, properly locating or positioning the clamp 14 on the sleeve 12. An O-ring seal or the like 18 may be positioned on the sleeve 12 to ensure a liquid-tight connection between the barb 17 and the sleeve 12. The configuration of this assembly avoids sterility breaks despite the insertion of an intrusive reusable sensor. In some embodiments the probe or tube that may be introduced into the sleeve 12 may be threaded (e.g., with PG13.5 or other thread configuration) or may be mechanically fastened to the sleeve 12.

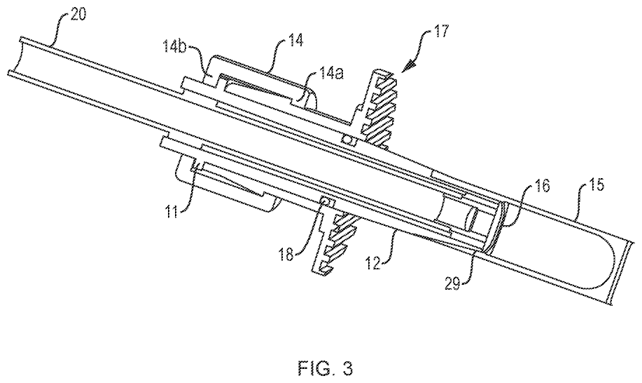

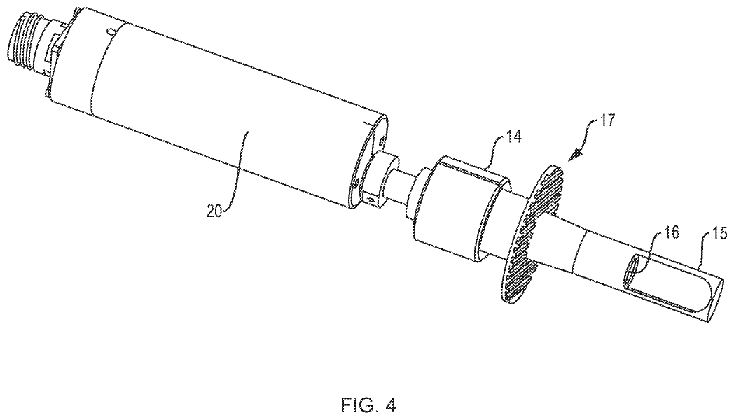

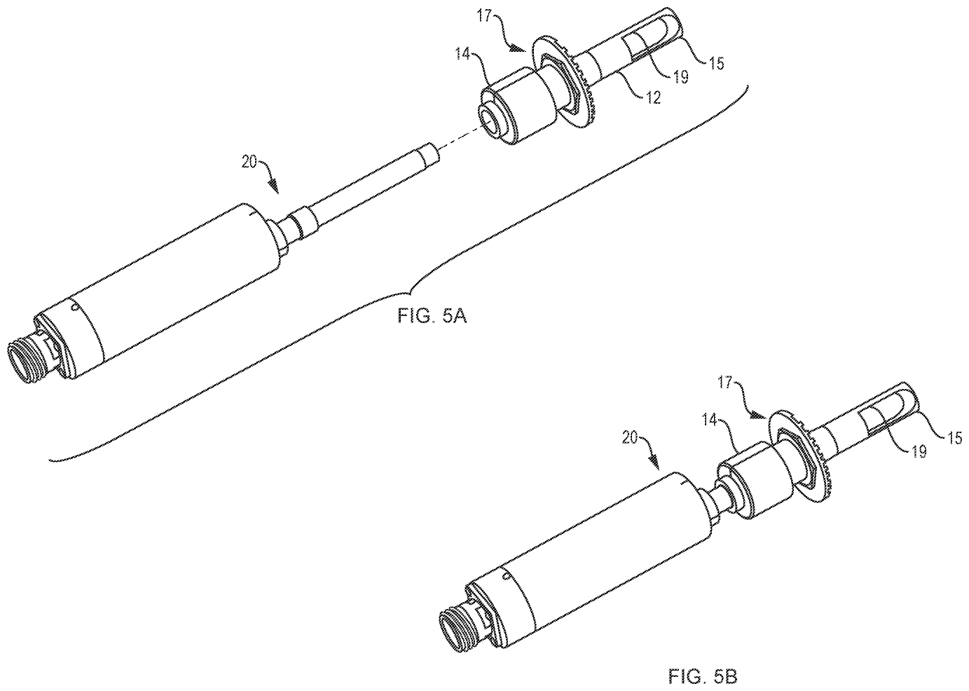

FIGS. 3, 4, 5A and 5B illustrate an embodiment of a probe 20 shown insertable or inserted into the sleeve 12. In the embodiment shown, the probe 20 includes a terminal optical lens 29 that positions next to the optical window 16 of the sleeve 12 when in the assembled condition.

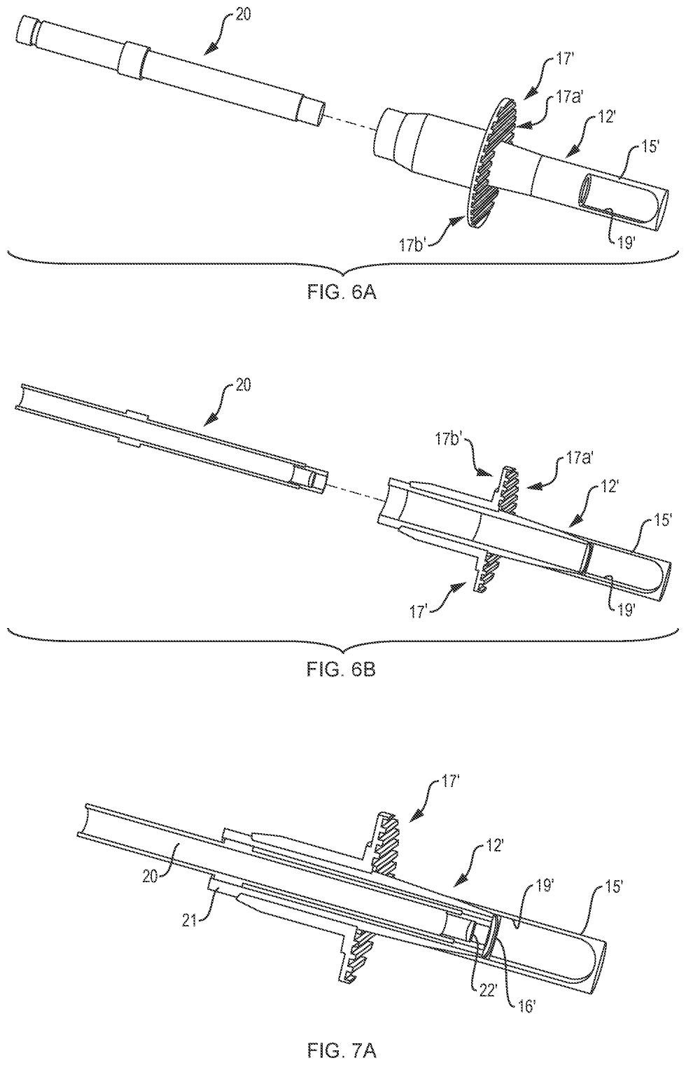

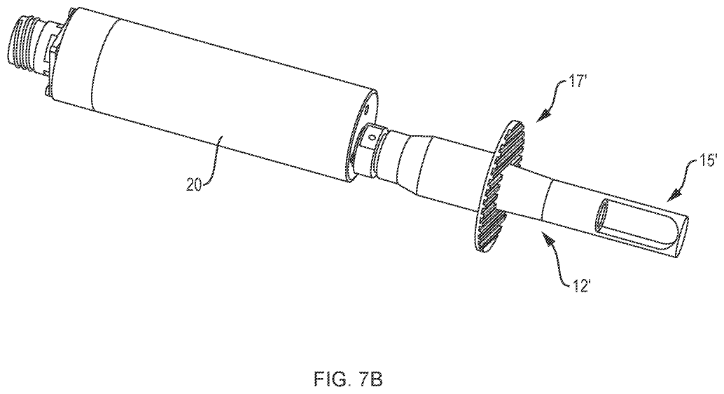

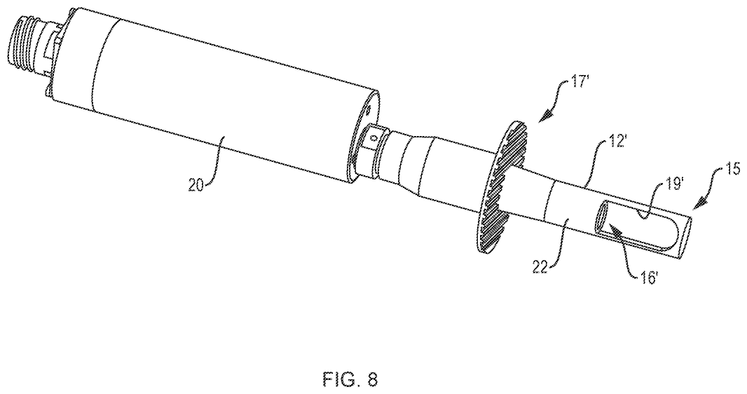

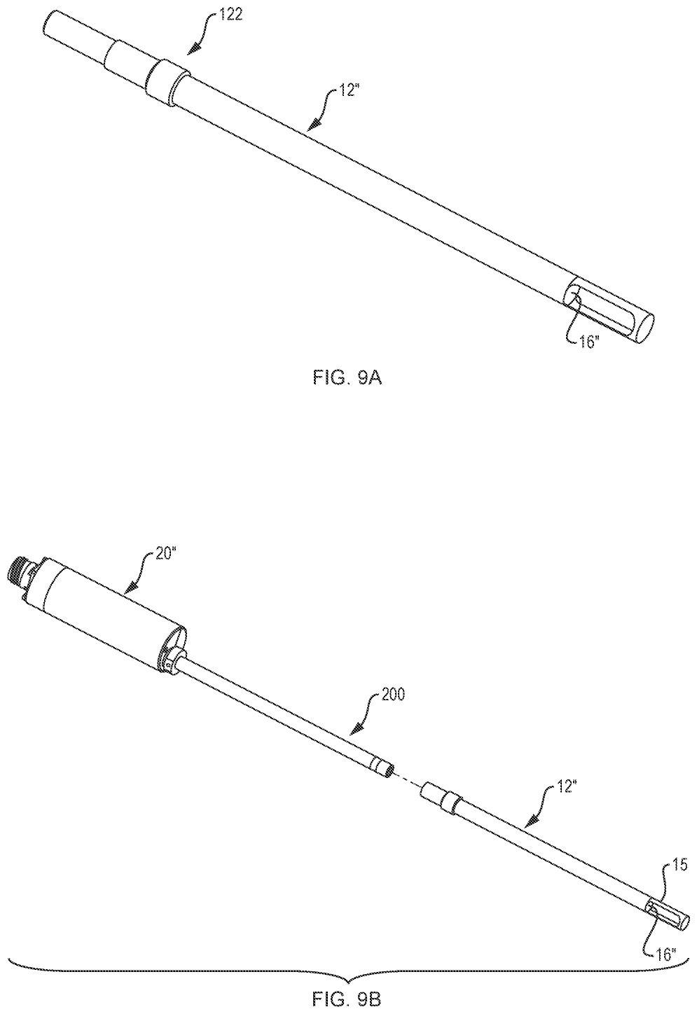

FIGS. 6A, 6B, 7A, 7B and 8 illustrate an embodiment where a barb-like fitting 17′ is integrated with a sleeve 12′: i.e., is formed as a single, integral piece. Thus, in certain embodiments, sleeve 12′ having an optical window 16′ (e.g., sapphire) overmolded on the sleeve 12′ and optionally one or more optical lenses, includes a barb-like structure 17′ overmolded on the sleeve 12′. Like the barb 17, the barb-like structure 17′ includes a first region 17A′ such as a flange that in use is positioned in the bioreactor chamber or volume: i.e., is internal thereto, and a second region 17B′ such as a stem with one or more radial ridges or serrations and is external to the bag: e.g., a probe insertion side. The flange encircles and is radially outwardly projecting from the stem. The sleeve 12′ has a generally cylindrical configuration and an internal bore or passageway configured (e.g., shaped and dimensioned) to receive a probe, such as a Raman probe. An optional end cap 15′ may be present for stray light management (e.g., reducing the ambient light intensity which can be detrimental to a Raman signal) and laser safety, and may be integrated in the sleeve 12′. As with the embodiment of FIG. 1, the flange functions as the interface with the bioreactor bag such as via port in the bioreactor.

In some embodiments the probe 20, which may include an internal stainless steel tube, may be coupled to the sleeve, once in its operable position, such as with external threads 21 that mate with internal threads in the sleeve. One or more internal optical lenses 22 may be mounted on the internal tube such as via a suitable lens holder. Alternatively, one or more optical lenses may be mounted in the sleeve 12′, and the probe body 20 may be mounted directly on the sleeve 12′ and be devoid of the internal tube.

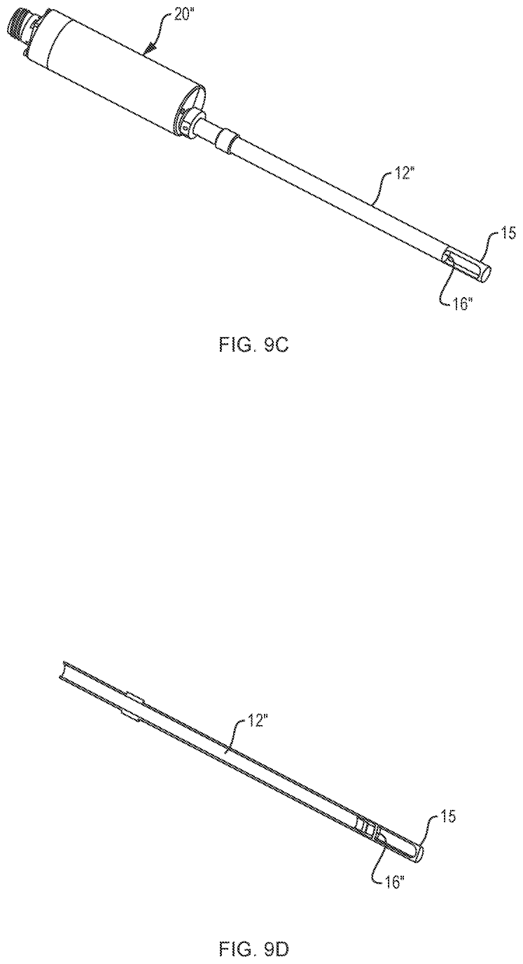

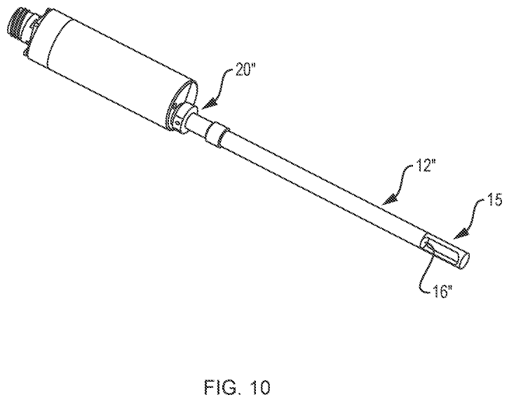

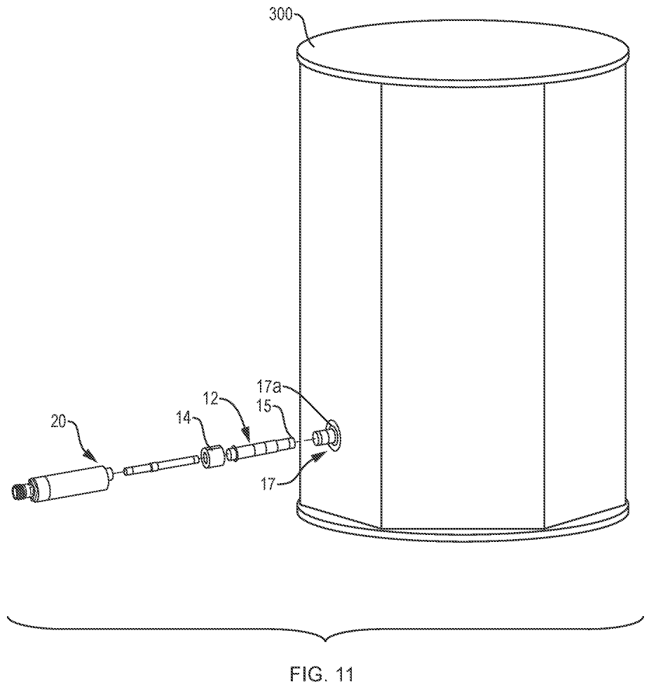

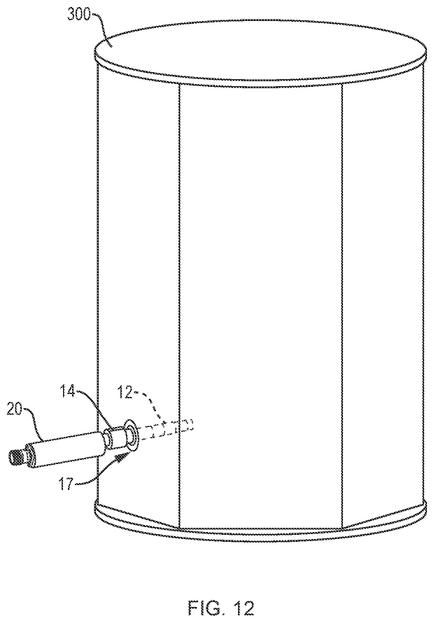

FIGS. 9A, 9B, 9C and 9D illustrate another embodiment where the proximal end of the single use sleeve 12″ with optical window 16″ includes external threads 122 (e.g., type PG13.5/M18 threads) that mate with threads on the port of the bioreactor, thereby eliminating the barb fitting. The probe 20″, having an internal tube 200 with an optical lens, may be inserted into the internal bore or passageway of sleeve 12″ (FIG. 9C). In some embodiments, the internal tube 200 could be eliminated, and the probe 20″ mounted directly to the single use sleeve 12″ (FIG. 10).

What is claimed is:

1. An interface assembly configured for insertion of a probe into a housing, said interface assembly comprising an elongated sleeve having a passageway configured to receive said probe, said sleeve containing an optical window; and a clamp for fastening said sleeve to a fitting positionable in a port of said housing.

2. The interface assembly of claim 1, wherein said sleeve has a proximal end where said probe is inserted, and a distal end spaced from said proximal end, and wherein a cap is positioned at said distal end.

3. The interface assembly of claim 2, wherein said cap extends longitudinally from said window.

4. The interface assembly of claim 1, wherein said cap has one or more radial openings.

5. The interface assembly of claim 1, wherein said probe includes an optical lens positioned next to said window of the sleeve 12 when in the assembled condition.

6. The interface assembly of claim 1, further comprising a barb fitting having a barbed stem that secures to said clamp, and a flange configured to be positioned in an interior volume of said housing.

7. The interface assembly of claim 1, wherein said housing has a threaded port, and wherein said interface assembly threads to said threaded port.

8. The interface assembly of claim 1, wherein said elongated sleeve is single-use.

9. The interface assembly of claim 1, wherein said optical window comprises sapphire.

10. The interface assembly of claim 1, wherein said housing is a bioreactor.

11. The interface assembly of claim 1, wherein said housing is a bioreactor bag.

12. A housing having an internal volume, the housing comprising a port providing access to said internal volume from a region external to said housing, and a fitting attached to said port, said fitting being configured to receive an interface assembly for insertion of a probe into said volume through said port, said interface assembly comprising an elongated sleeve having a passageway configured to receive said probe, said sleeve containing an optical window.

13. The housing of claim 12, wherein said interface assembly further comprises a clamp for fastening said sleeve to said fitting.

14. The housing of claim 12, wherein said fitting is a barbed fitting.

15. The housing or claim 12, wherein said fitting is threaded to said port.

16. The housing of claim 12, wherein said optical window comprises sapphire.

17. The housing of claim 12, wherein said housing is a bioreactor.

18. The housing of claim 12, wherein said housing is a bioreactor bag.
